(19) United States
(12) Patent Application Publication
(10) Pub. No.: US 2002/0028990 A1
(43) Pub. Date: Mar. 7, 2002

(54) Device and method for monitoring arterial oxygen saturation

(76) Inventors: John M. Shepherd, San Antonio, TX (US); Steven C. Walker, Baldwin, MO (US); John G. Alexander, O'Fallon, IL (US)

Correspondence Address: OFFICE OF THE STAFF JUDGE ADVOCATE, U.S. ARMY MEDICAL RESEARCH AND MATERIEL COMMAND, ATTN: MCMR-JA (MS. ELIZABETH ARWINE), 504 SCOTT STREET, FORT DETRICK, MD, 21702-5012, US

(21) Appl. No.: 09/905,991

(22) Filed: Jul. 17, 2001

Related U.S. Application Data
(63) Continuation-in-part of application No. 09/389,352, filed on Sep. 3, 1999, now Pat. No. 6,263,223.
(63) Continuation-in-part of application No. 09/780,570, filed on Feb. 12, 2001.
(60) Provisional application No. 60/099,579, filed on Sep. 9, 1998.
(60) Provisional application No. 60/101,079, filed on Sep. 18, 1998.
(60) Provisional application No. 60/101,143, filed on Sep. 18, 1998.
(60) Provisional application No. 60/182,018, filed on Feb. 11, 2000.

(51) Int. Cl. A61B 5/00

(52) U.S. Cl. 600/340

(57) ABSTRACT

A device for taking reflectance oximeter readings within the nasal cavity and oral cavity and down through the posterior pharynx. The device preferably includes a reflectance pulse oximeter sensor that preferably is resistant to bodily fluids to contact one of these capillary beds for the taking of readings and then for sending of these readings to a spectrophotometer or like device.

This application is a continuation-in-part application of U.S. application Ser. No. 09/389,352, filed Sep. 3, 1999, which claims the benefit of U.S. provisional Application Ser. No. 60/099,579, filed Sep. 9, 1998, U.S. provisional Application Ser. No. 60/101,079, filed Sep. 18, 1999, and U.S. provisional Application Ser. No. 60/101,143, filed Sep. 18, 1998; and is a continuation-in-part application of U.S. application Ser. No. 09/780,570, filed Feb. 12, 2001, which claims the benefit of U.S. provisional Application Ser. No. 60/182,018 filed on Feb. 11, 2000. Each of these applications is hereby incorporated by reference.

### FIELD OF THE INVENTION

This invention is directed to an apparatus for measuring blood oxygenation in multiple internal areas of a subject through either the nasal cavity or the oral cavity. More particularly, the invention relates to apparatus having pulse oximeter sensors to perform reflective pulse oximetry internal to a subject in these cavities.

### BACKGROUND OF THE INVENTION

With a few exceptions, tradition and technology have favored transillumination pulse oximetry in the operating theater. The principle of operation of the pulse oximeter is fairly simple but is arguably the most important development in anesthesia monitoring in the twentieth century. Two wavelengths of light (usually 660 nm and 940 nm) are used to spectrophotometrically determine the ratio of oxidized to reduced hemoglobin noninvasively as well as to determine the pulsatility of blood plethysmographically.

However, reflectance oximetry rather than transillumination oximetry was the earliest investigative form of the technique for taking oximeter readings. Transillumination pulse oximetry, without question, is the most effective form when oximetry is obtained through skin. However, when skin is not interposed as a barrier to capillary bed access, reflectance pulse oximetry easily can be achieved with very accurate results. Indeed, it is used commonly and effectively among intrapartum and neonatal patients whose capillary beds are easily accessed through their skin. The technique has also been applied to adult and pediatric burn patients by placing the reflectance sensor in wounds or over hyperemic sites such as healed partial thickness burns. The effect is achieved by the backscattering of incident bispectral light that traverses and, on reflection from nonabsorptive collagenous tissues, retraverses formed elements in the blood back to the oximeter elements. Rather than superseding transillumination pulse oximetry, this technique broadens the scope of possible monitoring sites, adding to the clinician's armamentarium.

Presently, the most common application of this in a medical setting is via transillumination through the capillary bed of a peripheral digit. However, young patients such as babies are apt to remove or reject foreign objects such as finger oximeters or inserted tubes upon realizing their placement when recovering from anesthesia or awaking from sleep. Sick children, in particular, are more likely to be restless and easily agitated and thus will resist any attempts to have medical readings taken like temperature or oximeter readings. Additionally, it is not unusual for multitrauma and thermally injured patients to either have severe peripheral vasoconstriction or to have severely damaged (or missing due to amputation) peripheral vascular beds.

There are other often overlooked capillary beds readily accessible in most adult burn patients and young children that are as amenable to reflectance oximetry similar to the forehead of the premature infant. The buccal surface, posterior soft palate, hard palate, lingual surfaces, and gums of a burned patient and/or child are seldom compromised no matter how severe the burn, and the capillary beds are very close to the surface in those areas. Transillumination pulse oximetry of the tongue and cheek has been documented as a viable method of monitoring, but not everyone has the equipment available to place a transilluminating pulse oximeter on the tongue or cheek.

Recent studies indicate that oral pulse oximetry is a superior modality when compared to peripheral transillumination pulse oximetry. A variety of studies have shown that oral pulse oximeters are more reliably and rapidly responsive than peripheral pulse oximeters. However, these studies use oral transillumination pulse oximetry, held in place via complex devices or pieces of improvised malleable metal. Oral secretions, equipment failure, and placement difficulty often render these techniques ineffective.

Prior pulse oximeter sensors inserted through the mouth are usable only when the patient is under general anesthesia. These pulse oximeter sensors are inserted to reach the larynx area, for example, U.S. Pat. No. 5,282,464 to Brain et al. Another known method uses transillumination pulse oximetry of the posterior tongue, but this method possibly may not be used with a patient, who is awake, for example, U.S. Pat. No. 5,205,281 to Buchanan. Also, the posterior tongue is not the most accessible body part to take oximetry measurements.

Notwithstanding the usefulness of the above-described devices, and the above-identified recognized viability of transilluminating pulse oximetry, a need exists for a more convenient method for obtaining oximeter readings from a subject.

### SUMMARY OF THE INVENTION

The invention while addressing the problems of the prior art obtains advantages that were not achievable with the prior art apparatuses and methods.

According to one aspect of the present invention, a reflectance pulse oximeter sensor for use within an intrahead cavity site of a subject preferably includes means for performing reflectance pulse oximetry measurements from a palate, and means for communicating with an external device.

According to one aspect of the present invention, a reflectance pulse oximeter sensor for use within an intrahead cavity site of a subject preferably includes oximeter sensor elements, wherein at least one of said oximeter sensor elements receives reflected light originating with another of said oximeter sensor elements, and means for causing contact to occur between said oximeter sensor elements and a palate.

According to one aspect of the present invention, a reflectance pulse oximeter sensor for use within an intrahead cavity site of a subject preferably includes means for performing reflectance pulse oximetry measurements from a proximal pharyngeal surface, and means for communicating with an external device.

According to one aspect of the present invention, a reflectance pulse oximeter sensor for use within an intrahead cavity site of a subject preferably includes oximeter sensor elements, wherein at least one of said oximeter sensor elements receives reflected light originating with another of said oximeter sensor elements, and means for causing contact to occur between said oximeter sensor elements and a proximal pharyngeal surface.

According to one aspect of the present invention, a reflectance pulse oximeter sensor for use within an intrahead cavity site of a subject preferably includes means for performing reflectance pulse oximetry measurements from a gingival surface, and means for communicating with an external device.

According to one aspect of the present invention, a reflectance pulse oximeter sensor for use within an intrahead cavity site of a subject preferably includes oximeter sensor elements, wherein at least one of said oximeter sensor elements receives reflected light originating with another of said oximeter sensor elements, and means for causing contact to occur between said oximeter sensor elements and a gingival surface.

According to one aspect of the present invention, a reflectance pulse oximeter sensor for use within a nasal cavity of a subject preferably includes means for performing reflectance pulse oximetry measurements from a capillary bed in the nasal cavity, and means for communicating with an external device.

According to one aspect of the present invention, a reflectance pulse oximeter sensor for use within a nasal cavity of a subject preferably includes oximeter sensor elements, wherein at least one of said oximeter sensor elements receives reflected light originating with another of said oximeter sensor elements, and means for causing contact to occur between said oximeter sensor elements and a capillary bed in the nasal cavity.

An object of this invention is to provide an effective device and method for taking pulse oximetry measurements from nasal, oral, and posterior pharyngeal capillary beds.

Another object of the invention is the use of reflectance pulse oximetry via the nasal and/or oral cavities for a variety of surgical, anesthetic, or critical care procedures or situations to include patients that are awake, sedated, undergoing general anesthesia, or undergoing general anesthesia.

Another object of the invention is to allow for lingual placement of a pulse oximeter sensor for reflectance readings to provide efficient and clinically accurate pulse oximetry measurements.

Another object of the invention is to allow for buccal placement of a pulse oximeter sensor for reflectance readings to provide efficient and clinically accurate pulse oximetry measurements.

Yet another object of the invention is to monitor oxygen levels in newborns and young children who are difficult to monitor because of their natural restlessness and young age.

Still another object of the invention is to monitor oxygen levels in severely burned ICU patients who are difficult to monitor.

An advantage of the invention is an improvement in the quality of care resulting from using a straightforward method with easily used devices to take internal oximetry measurements and readings.

Another advantage of the invention is that EMS crews and personnel will be able to use this invention easily in the field during, for example, emergency situations.

Another advantage of the invention is improved pulse oximetry readings.

Another advantage of the invention is reflectance pulse oximetry requires less power to function and thus less heat is produced than transilluminance pulse oximetry. The decrease in produced heat lowers the risk the subject will be burned.

Yet another advantage of the invention is that ambient light will not degrade the oximeter readings because the invention is within the mouth of a subject.

The invention accomplishes the above objectives and achieves the advantages. The invention is easily adapted to a wide variety of situations.

Furthermore, intraoral buccal, lingual, proximal posterior pharyngeal, or palatal placement of a pulse oximeter probe in a configuration relying upon reflected light will provide pulse oximetry measurements comparable to those obtained by peripheral pulse oximetry. Test protocols suggest that buccal, lingual, proximal posterior pharyngeal, and palatal reflectance pulse oximetry provides a simple, accurate means of monitoring arterial oxygen saturation in the severely burned patient where oximetric monitoring presents a challenge.

Furthermore, the invention is extremely useful in cases where it is difficult at best or not even possible to attach prior art pulse oximeter sensors with clips or straps to the patient. The types of patients that this invention would be useful with are critically ill or injured patients including newborns, babies, young children, animals, or burn or trauma patients without alternative sites and maxillofacial injuries.

Given the following enabling description of the drawings, the invention should become evident to a person of ordinary skill in the art.

### DESCRIPTION OF THE DRAWINGS

The use of cross-hatching within these drawings should not be interpreted as a limitation on the potential materials used for construction. Like reference numerals in the figures represent and refer to the same element or function.

FIG. 1 illustrates a flowchart illustrating the steps for performing the preferred embodiment.

FIG. 2 depicts a nasopharyngeal airway oximeter sensor being used in the method against the proximal posterior pharynx.

FIG. 3 illustrates an oral airway oximeter sensor being used in the method against the proximal posterior pharynx.

FIG. 4(a) depicts the oral airway oximeter sensor being used in the method against a buccal surface.

FIG. 4(b) illustrates the oral airway oximeter sensor being used in the method against the lingual surface.

FIG. 5(a) depicts a pacifier oximeter sensor being used against the lingual surface.

FIG. 5(b) illustrates the pacifier oximeter sensor being used against the hard palate.

FIG. 6 depicts the nasopharyngeal airway oximeter sensor.

FIG. 7 illustrates a bottom view of the nasopharyngeal airway oximeter sensor.

FIG. 8 depicts a radial cross-section view of the nasopharyngeal airway oximeter sensor.

FIG. 9 illustrates a rear view of the nasopharyngeal airway oximeter sensor.

FIG. 10 depicts a side view of the oral airway oximeter sensor.

FIG. 11 illustrates a front view of the oral airway oximeter sensor.

FIG. 12 depicts a rear view of the oral airway oximeter sensor.

FIG. 13 illustrates a top view of the oral airway oximeter sensor.

FIG. 14 depicts a side view of another oral airway oximeter sensor.

FIG. 15 depicts a partial front view of the distal end of the oral airway oximeter sensor illustrated in FIG. 14.

FIG. 16 depicts a side view of the pacifier oximeter sensor.

FIGS. 17(a)-(i) illustrate side cross-sections of different nipples for the pacifier oximeter sensor to illustrate various alternative placements and arrangements of pulse oximeter elements.

FIGS. 18(a)-(f) illustrate top views of various alternative placements and arrangements of the pulse oximeter elements in the pacifier oximeter sensor.

FIGS. 19(a) and (b) illustrate cross-sections of examples of attaching the nipple to the shield for the pacifier oximeter sensor.

FIGS. 20(a)-(b) depict an example of a shield structure for use with the pacifier oximeter sensor.

FIG. 21 illustrates a top view of an alternative pacifier oximeter sensor.

FIG. 22(a) depicts a top cross-section of another alternative pacifier oximeter sensor.

FIG. 22(b) illustrates a rear view of the alternative pacifier oximeter sensor illustrated in FIG. 22(a).

FIG. 22(c) depicts a block diagram for the alternative pacifier oximeter sensor illustrated in FIGS. 22(a) and (b).

FIG. 23 illustrates a top view of another alternative pacifier oximeter sensor.

### DETAILED DESCRIPTION OF THE INVENTION

a. Overview

In accordance with the present invention, there are apparatuses to take oximeter readings from different sites within a subject, which may be either human or animal, for the purposes of determining the amount of oxygen within the blood of the subject. The oximeter readings are accomplished using reflectance oximetry from capillary beds that are readily accessible within the subject. The capillary beds include, for example, the nasal mucosa, the nasal passageway, the proximal posterior pharynx, the hard palate, the soft palate, the superior lingual surface, the inferior lingual surface, the gingivae, the mouth floor, and the buccal surface. Each of these capillary beds is accessible through at least one opening in the subject's head leading to an intrahead cavity, which includes an oral cavity and a nasal cavity. The oral cavity extends from the lips to the oral portion of the proximal posterior pharynx, i.e., pars oralis. The nasal cavity extends from the nostrils to the nasal portion of the proximal posterior pharynx, i.e., pars nasalis. The nasal cavity and the oral cavity together form what is known as the nasal oral pharynx region of an individual. The term proximal when used with posterior pharynx and pharyngeal surface indicates the curved region at the top of throat.

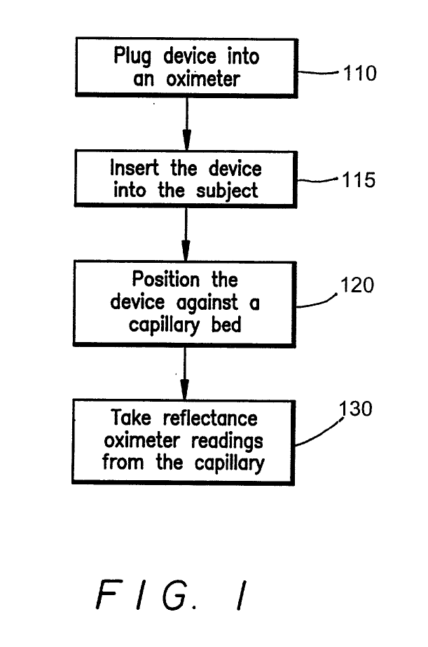

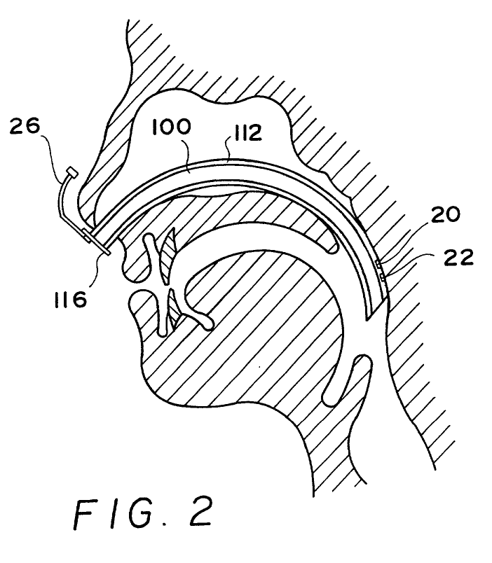

FIG. 1 illustrates a flowchart showing the common steps for taking oximeter readings from the previously mentioned capillary beds. In the first step 110, which may actually occur at a later point but no latter then the initiation of taking pulse oximeter readings, the pulse oximeter sensor elements are connected to an oximeter device such as a spectrophotometer. In step 115, the oximeter sensor device is inserted into the subject through an available opening such as the mouth or the nose. In step 120, the pulse oximeter sensor is positioned adjacent the relevant capillary bed such that the pulse oximeter sensor elements are facing and preferably in contact with the capillary bed to allow for reflectance pulse oximetry. The placement of a device with a pulse oximeter sensor is illustrated, for example, in FIGS. 2-5(b). In step 130, reflectance pulse oximeter readings are taken from the relevant capillary bed. While taking the pulse oximeter readings, the pulse oximeter sensor elements preferably remain in contact with the relevant capillary bed to continue the flow of accurate oximeter readings.

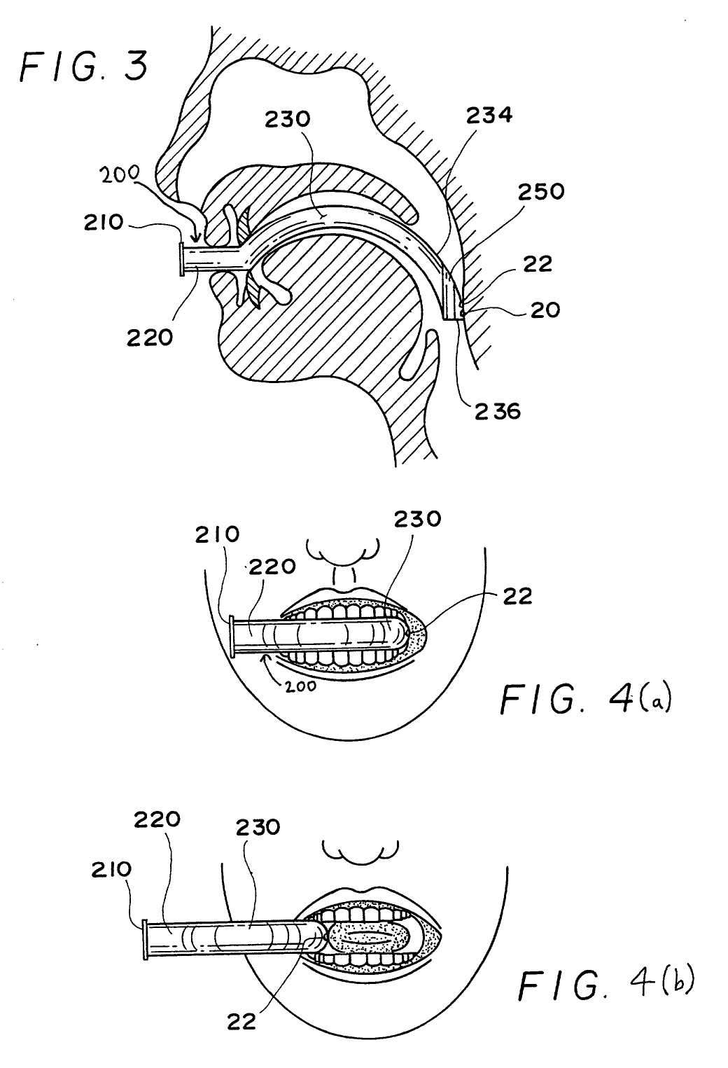

In the case where the capillary bed is the nasal mucosa, the opening through which the pulse oximeter sensor is inserted is one of the nostrils as shown in FIG. 2. In the case where the capillary bed is the posterior pharynx, the pulse oximeter sensor may be inserted through either the nasal cavity or orally depending of the type of device used as exemplified by the devices shown in FIGS. 2 and 3. If the capillary bed is the soft or hard palate, the superior lingual, or the buccal surface, then the pulse oximeter sensor preferably is inserted into the mouth as shown, for example, in FIGS. 3-5(b).

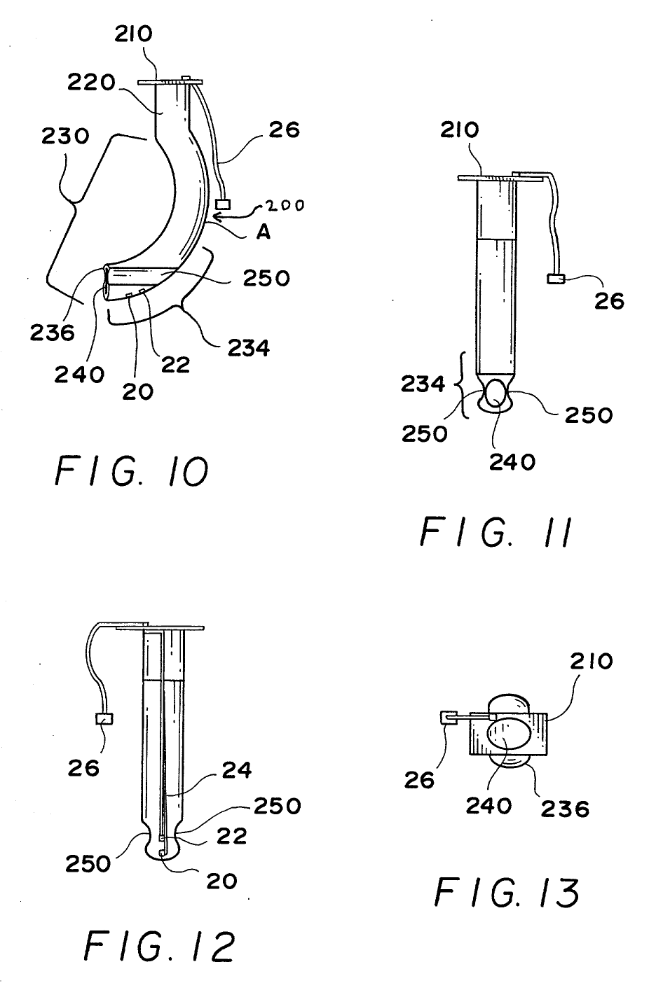

The readings may be taken with a variety of devices, some of which are discussed below and shown in FIGS. 2-23. Suitable devices for performing the method preferably include at a minimum, the following: pulse oximeter sensor elements, including a light source 20 and a light detector 22, and a support surface or structure (means for causing contact) 100, 200, 412. The pulse oximeter sensor elements 20, 22 are arranged to perform reflectance pulse oximetry and in such an arrangement are means for performing reflectance pulse oximetry measurements. In addition, the devices may include wiring 24 and an external cord 26, both of which may be connected to the pulse oximeter sensor elements 20, 22.

More particularly, in the exemplary devices, the pulse oximeter sensor elements include a light source 20, which preferably emits light with wavelengths of 660 nm (red) and 940 nm (near infrared), and a light detector 22. Although, the light source 20 may emit light of a variety of wavelengths typically emitted in light sources of pulse oximeter sensors. The placement and location of the light source 20 and the light detector 22 depicted in the Figures may be switched with respect to each other.

The light source 20 emits at least two frequencies of light at, for example, about 660 nm and about 940 nm. The light source 20 preferably is one or more of the following: two light emitters such as light emitting diodes (LED), a bispectral emitter, a dual spectral emitter, a photoemitter, or a semiconductor die. However, any light source that facilitates reflectance pulse oximetry may be employed. Typically, the two emitter arrangement will include a red LED emitting light with a wavelength around or at 660 nm and a near-infrared LED emitting light with a wavelength in the range of 890 to 950 nm and more particularly at about 940 nm. The light source 20 may emit light having a bandwidth, for example, in the range of 20 to 50 nm.

The light detector 22 detects light emitted by the light source 20. Electrical signals representing the detected light are transmitted by the light detector 22 to a spectrophotometer, or other similar oximeter device, that discriminates between the relative intensity of these emissions and provides an index as to the degree of oxygen saturation of hemoglobin in blood. Preferably, the light detector 22 may be one of the following: a photoelectric receiver, a photodetector, or a semiconductor die.

Wiring 24 includes conductive lines and contact electrodes. An external cord 26 preferably is insulated and connects to the wiring 24 at the proximal end of the support structure so that the external cord 26 preferably is either outside the subject or near the opening in which the device was inserted. The external cord 26 has a standard plug design to interface with a pulse oximetry spectrophotometer, a pulse monitor such as a plethysmograph, or other external device. The spectrophotometer provides the electrical signals for controlling the pulse oximeter elements 20 and 22. The light source 20 and the light detector 22 may be in wireless communication (an exemplary structure is discussed later) with the external device instead of connected with the external cord 26 shown, for example, in FIG. 3, 14, and 15. Alternatively, the external cord 26 may be a jack to connect to a reusable cable such as the cable sold with the Nellcor® OxiCliq® systems (Mallinckrodt, Inc., St. Louis, Mo., U.S.A.). The above-described and alternative ways (including wired and wireless connections) are possible means for communicating with an external device.

The apparatus according to the invention may be used in a variety of surgical, anesthetic, combat or critical care procedures or situations that include patients that are awake, sedated or undergoing general anesthesia. In particular, the apparatus may be used throughout the pre-induction, throughout induction, during, upon emergence from, and after anesthesia without switching devices. This advantage is accomplished while avoiding uncomfortable stimulation deep in the throat, thus minimizing the possibility of gagging, vomiting, aspiration, and impingement of the teeth upon the endotracheal tube prior to extubation.

The following devices are capable of being used in conjunction with the method for at least one of the previously discussed capillary beds. With each device there is an explanation as to how the device is utilized in conjunction with the method.

b. Nasopharyngeal Airway Oximeter Sensor

The first device is a nasopharyngeal airway with a reflectance pulse oximeter sensor that is illustrated in FIGS. 2 and 6-9. This device preferably includes a nasopharyngeal airway 100 in combination with pulse oximeter elements 20 and 22, wiring 24, and external cord 26. The pulse oximeter sensor elements 20 and 22 and wiring 24 are preferably disposed within the wall of the nasopharyngeal airway 100.

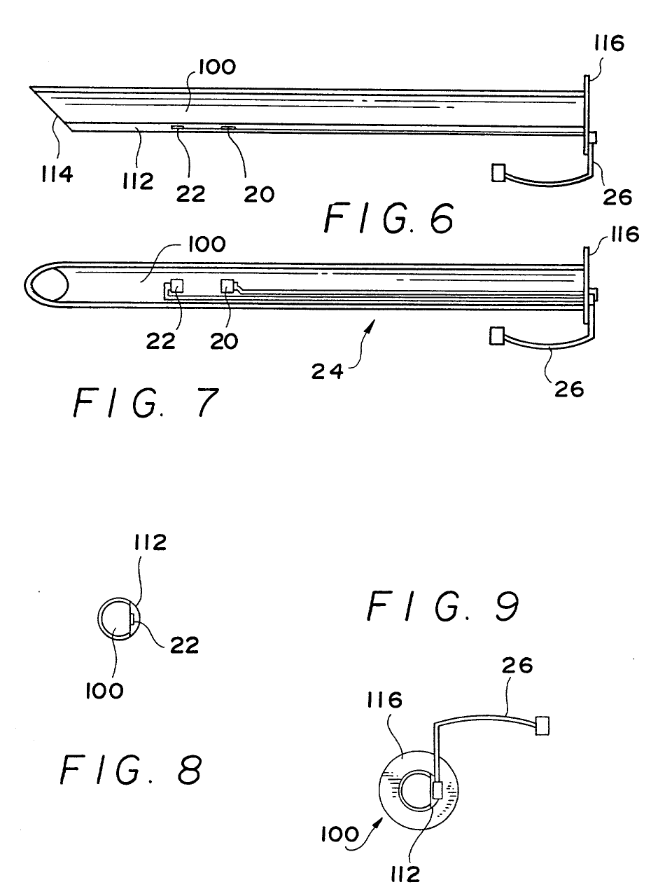

As depicted in FIG. 6, the nasopharyngeal airway 100 is preferably a hollow, elongated member defining a passageway, e.g., a cylindrical tubular member, having an insertion end 114 and a base end (or proximal end) 116. The insertion end 114 preferably is angled. The base end 116 may be flat and disposed substantially perpendicular to the rest of the nasopharyngeal airway or angled. The base end 116 may include a notch or other marking corresponding to the tip of the nasopharyngeal airway 100 to assist the user in further positioning the device in the patient after insertion of the device. Alternatively, the base end 116 may be omitted.

Preferably, the wall of the nasopharyngeal airway 100 is made of a clear polymer. Furthermore, it is preferable that the wall may include a thickened section 112 around approximately one-third of the cross-sectional circumference to house the pulse oximeter sensor elements 20 and 22 and wiring 24. Alternatively, the thickened section 112 may be around just the elements and not run the length of the nasopharyngeal airway 100. The pulse oximeter sensor elements 20 and 22 and wiring 24 are preferably embedded and sealed in the wall of the nasopharyngeal airway with a cover, which may be integrally formed with the nasopharyngeal airway 100, protecting them. Preferably, the cover is a clear, fluid impermeable plastic.

Alternatively, the pulse oximeter sensor elements 20, 22, and 24 may be disposed within the passageway of the nasopharyngeal airway 100. A disposable pulse oximeter like the Nellcor® Oxisensor® II N-25 or D-25 (Nellcor Puritan Bennett®, Inc., Pleasanton, Calif.) may be stripped of its surroundings to leave only the pulse oximeter sensor elements. The pulse oximeter elements may then be fed into the nasopharyngeal airway 100 along one side of the passageway. Even though the pulse oximeter sensor elements and wiring may be present in the passageway, there is sufficient airflow capacity to supply adequate oxygen to the patient.

Preferably, the pulse oximeter sensor elements 20 and 22 are located near the angled end 114 to facilitate readings being taken from the proximal posterior pharynx as shown in FIG. 2. This arrangement provides for easy access and a reliable contact point. However, if the pulse oximeter sensor elements 20 and 22 are located near the base end 116, then the readings will be taken from within the nasal cavity. The nasal cavity, while adequate, provides less reliable and accurate pulse oximetry readings than the posterior pharynx.

The nasopharyngeal airway preferably is manufactured using polypropylene, polyvinyl chloride, silicones, epoxies, polyester, thermoplastics, rubber, similar flexible material, etc. The material should be sufficiently flexible that it freely bends in accordance with the contour of the nasal passageway. The thickened area is preferably formed from a clear or semi-translucent material to allow for the passage of light from the light source 20 and to the light detector 22.

In use, the nasopharyngeal airway 100 preferably takes measurements from a central measurement site, namely the proximal posterior pharynx, posterior soft palate or nasal mucosa. The data collected from these locations has proven more reliable then data attainable from periphery locations. The increased reliability is believed to be due to the centrality of the measurement location and the stability of the measurement surfaces.

This device is particularly useful when the patient is awake but sedated. However, the device may be used while the patient is fully awake, during induction of anesthesia, during general anesthesia, on emergence from anesthesia and during recovery. This device will allow pulse oximeter measurements to be taken in very wet environments as the oximetric components and wiring are sealed within the polymeric envelope of the nasopharyngeal airway. This device is useful for taking pulse oximeter measurements in the field and in emergency medical areas, because the device is capable of both establishing an airway and providing pulse oximetry monitoring in a single device.

c. Oral Airway/Bite Block Pulse Oximeter Sensor

Another device suitable for performing the method according to this invention is a pulse oximeter sensor combined with a combination oral airway and bite block as illustrated in FIGS. 3-4(b) and 12-15.

As depicted, the device 200 preferably includes a base 210, a straight portion 220, and a palatal and proximal pharyngeal contour portion 230 preferably arched to be physiologically compatible with the palate and pharynx. The airway also preferably includes oximeter elements 20 and 22, which reside in the posterior distal curvature of the device. As represented in the figures, the optional wiring 24 and external cord 26 are represented, but as discussed above may be removed in a wireless configuration. The contour portion 230 preferably includes arched section A having an outer distal curve 234 and a distal end 236. The contour portion 230 is preferably integrally formed with straight portion 220, which preferably includes a proximal end abutting the base 210. The base 210 preferably is large enough to allow the device to be manipulated by the user. Although, the base 210 may be omitted.

A central passageway or channel 240 may be formed within the device to extend from the distal end 236 to the base 210 (or the proximal end of the straight portion 220). As is apparent to one of ordinary skill in the art in view of the present disclosure, the cross-section and dimensions of the passageway 240 may be selected to maximize the airflow through the passageway without reducing the integrity of the device.

Preferably, the light source 20 and the light detector 22 are embedded in the body of the device along the outer distal curve 234 facing radially outward with a cover, which may be integrally formed with the airway, protecting them. Preferably the cover is a clear, fluid impermeable plastic. Alternatively, the pulse oximeter elements 20 and 22 may be disposed within passageway 240 adjacent the outer distal curve 234. FIGS. 3 and 10-13 depict wiring 24 connecting the pulse oximeter elements 20 and 22 to the external cord 22. The wiring 24 is preferably also embedded in the body of the contour portion 230.

The pulse oximeter elements 20 and 22 may be disposed in a variety of locations along the passageway in accordance with the desired application. Preferably, the pulse oximeter elements 20 and 22 are placed closer to the distal end 236 of the device so that the readings may be taken from the proximal posterior pharynx area, the buccal surface, the lingual surface, the gingiva surfaces, or the mouth floor of the patient. As the pulse oximeter elements 20 and 22 are moved towards the apex of the arched section A, the readings more likely will be taken from the soft palate of the patient. The dividing line between these regions is highly dependent on the internal dimensions of the patient. However, the readings obtained from each area work equally well in terms of accuracy. Also, the closer to the apex of the arched section A the pulse oximeter elements 20 and 22 are, the more difficult it is for the device to contact the buccal surface or the lingual surface when the device is used as a bite block. When the pulse oximeter elements 20 and 22 are positioned away from the apex of the arched section A towards the proximal end abutting the base 210, the readings will be taken from the hard palate, which also will provide accurate pulse oximetry readings.

Alternatively, bilateral grooves (or recesses) 250 into which the teeth may fit are preferably disposed in opposing relationship to each other and formed in the contour portion 230 to facilitate operation of the device as a bite block. More preferably, the bilateral grooves extend from the distal end 236 to a point along the outer distal curve 234. The bilateral grooves 250 may be filled with a sponge-like or soft material, e.g., foam or rubber to protect the teeth. The bite block groove/recess feature is not necessary for this device to be used for monitoring arterial oxygen saturation pursuant to the preferred method.

Alternatively, the pulse oximeter elements may be disposed in the passageway. A disposable pulse oximeter sensor like the Nelicor® Oxisensor® II N-25 may be stripped of its surroundings to leave only the pulse oximeter elements. The pulse oximeter elements are then fed along the topside of the passageway 240. Although the pulse oximeter elements and wiring may be present in the passageway 240, there will be sufficient airflow capacity in the passageway to supply oxygen to the patient. The N-25 pulse oximeter sensor when installed in this manner does not overdrive as a result of the emitted brightness from the light source, because of the optical effects provided by the oropharyngeal airway.

To facilitate operation of the device as a pulse oximeter sensor, a plastic bag, protective cover or similar item may be placed around the distal end 236. This addition is particularly useful when there is excess moisture that might interfere with the operation of the pulse oximeter sensor elements.

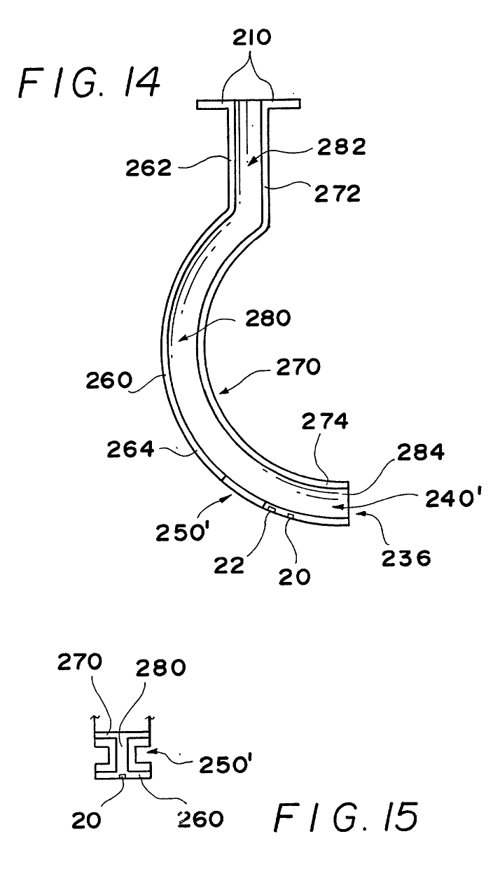

Alternatively, the oral airway/bite block oximeter sensor may have an I-beam construction as shown in FIGS. 14 and 15. The I-beam structure preferably includes a first wall 260, a second wall 270, and a third wall 280. Preferably, the first wall 260 runs parallel to the second wall 270 and the third wall 280 runs perpendicular to and between the first and second walls 260 and 270. Each wall preferably includes a straight portion 262, 272, and 282 and a distal curve portion 264, 274, and 284 configured to fit the contour of the palatal and proximal pharyngeal. At the end opposite the distal end 236 preferably is a base 210, although the base 210 may be omitted. A passageway 240′, as shown in FIG. 14, preferably is formed on either side of the third wall 280 and is framed by the first and second walls 30 and 40. A groove 250′ may be provided in the first wall 260 to provide a recess for the teeth to pass through to facilitate operation as a bite block.

Preferably, the pulse oximeter elements 20 and 22 are located within the first wall 260 in the distal curve portion 264. Preferably, the first wall 260 is thickened in the area around the pulse oximeter elements 20 and 22 slightly relative to the second wall 270 to better house the pulse oximeter elements 20 and 22. This area may include translucent material to allow for light to travel through the first wall 260. As one of ordinary skill in the art will appreciate, the pulse oximeter elements 20 and 22 may be placed within the third wall 280 in the distal curve portion 284 (not shown). The pulse oximeter elements 20 and 22 are positioned to perform reflectance pulse oximetry. The pulse oximeter elements 20 and 22 may be placed anywhere along the length of the first and third walls 260 and 280 in a manner similar to the first oral airway/bite block oximeter sensor discussed above. The wiring 24 connected to the pulse oximeter elements 20 and 22 preferably is within the same wall as the pulse oximeter elements 20 and 22.

The base oral airway/bite block structure is preferably manufactured using polypropylene material that is either molded or extruded. Molding will produce a more rigid structure than extrusion. The sponge-like material, e.g., foam or rubber in the recesses may be added after forming the base oral airway/bite block. Both molding and extrusion will allow the pulse oximeter sensor elements to be embedded in the oral airway/bite block structure.

To accomplish a change between the two modes, this device only needs to be repositioned within the patient thus avoiding the need to exchange devices as required with present devices. For example, during anesthesia, it may be desirable to use the device as an oral airway to establish a ventilatible airway for the patient. When used as such, the device is preferably inserted into the patient's mouth such that it impinges upon the posterior soft palate and/or the posterior pharynx along the outer distal curve 234.

In addition, before, on induction of, during, on emergence from and after anesthesia, it may be desirable to employ the device as a bite block. When used as such, the device is preferably inserted into the patient's mouth such that the bilateral grooves 250 on the sides of the contour portion 230 may be inserted between the molars and/or bicuspids on one side of the mouth. The outer distal curve 234 in this mode abuts the buccal mucosa, as shown in FIG. 4(a). The alternative preferred insertion method is to place the device such that the bilateral grooves 250 are inserted between the molars and/or bicuspids on the other side of the mouth so that the outer distal curve 234 will abut the lingual surface of the tongue, as shown in FIG. 4(b). Neither mode will stimulate the posterior tongue/pharynx.

d. Pacifier Pulse Oximeter Sensor

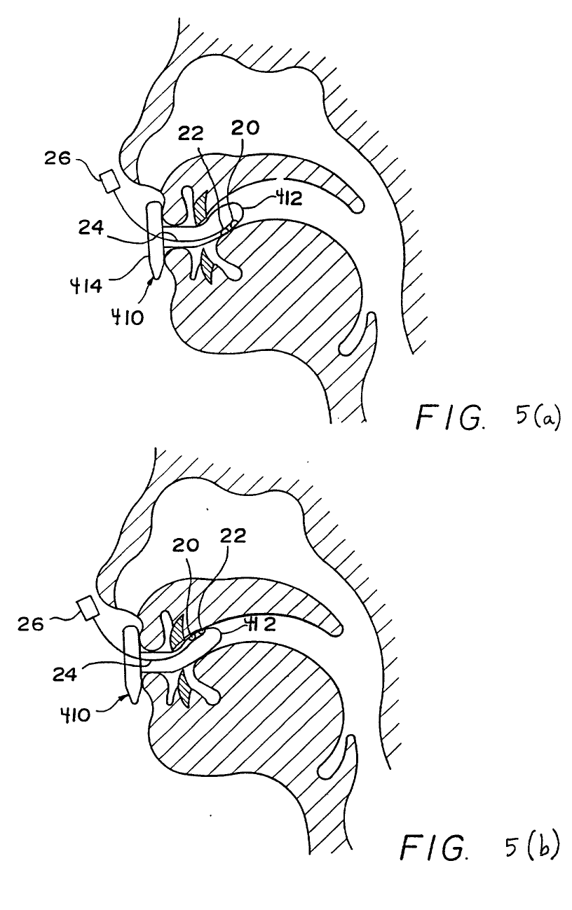

Another device suitable for performing the method according to this invention is a pulse oximeter sensor combined with a pacifier as illustrated in FIGS. 5(a)-(b) and 16-23.

FIGS. 16-23 illustrate a preferred embodiment and alternative component arrangements of the pacifier oximeter sensor assembly. The assembly preferably includes a pacifier 410, pulse oximeter sensor elements 20, 22, and wiring 24.

The pacifier 410 preferably includes a nipple (or baglet) 412 and a shield (or guard) 414. The nipple 412 may be a variety of shapes in addition to those shown in FIGS. 16-23 that will allow the subject to apply a suction force to the nipple 412. Exemplary shapes for the nipple 412 include orthodontic, bottle nipple, spherical, and thumb shaped. The nipple 412 preferably is a flexible material typically used to make pacifiers and baby bottle nipples such as polypropylene, polyvinyl chloride, silicones, epoxies, polyester, thermoplastics, rubber, or similar flexible material. Preferably, the material used to make the nipple 412 will be at least partially translucent to allow light to pass through in the area of the pulse oximeter sensor elements 20, 22. Preferably, the nipple 412 will have an inner cavity 4124 formed as a void in the nipple material 4122. However, the nipple 412 may be solid or filled with a flexible material to increase the protection of the pulse oximeter sensor elements 20, 22 and wiring 24.

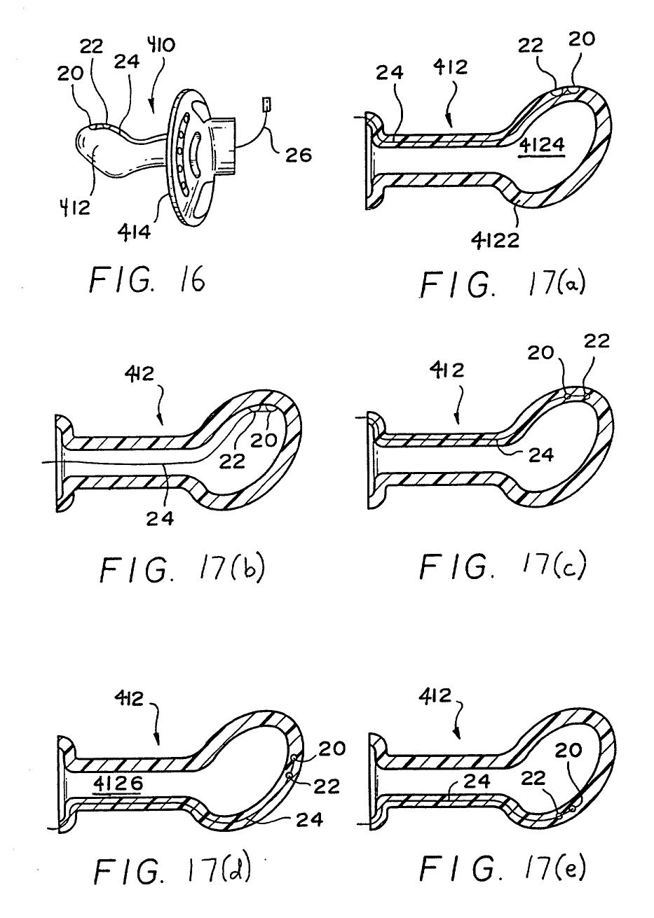

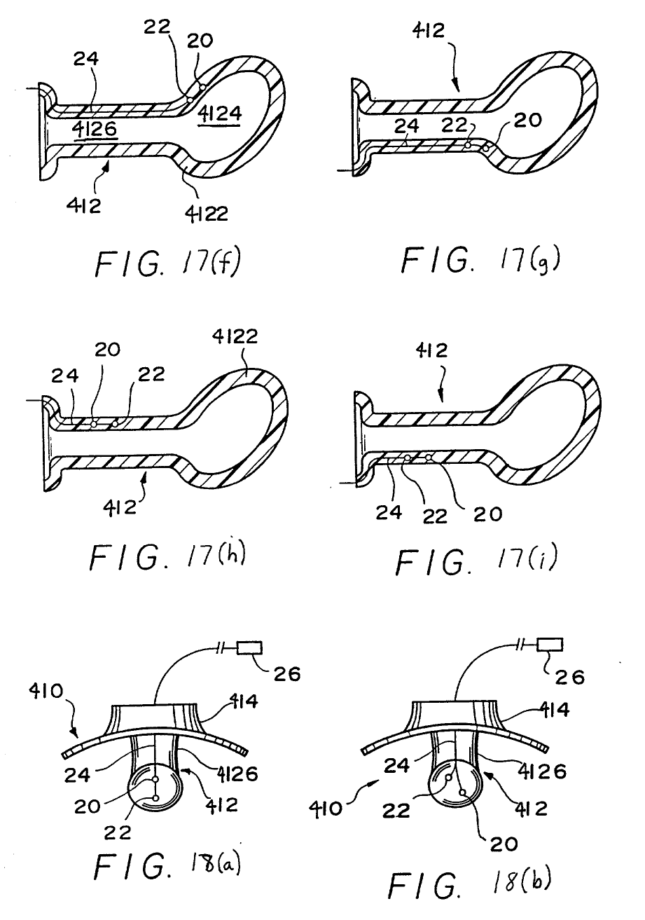

The pulse oximeter sensor elements 20, 22 preferably are within the material 4122 making up the nipple 412 to reduce the impact of the material 4122 on the transmission of light through the material 4122. However, the pulse oximeter sensor elements 20, 22 may be nested within the nipple material 4122 as shown, for example, in FIG. 17(c) or the pulse oximeter sensor elements 20, 22 may abut the nipple material 4122 on the inner cavity surface as shown, for example, in FIG. 17(b). The pulse oximeter sensor elements 20, 22 preferably will be placed in a position to transmit light and receive backscattered light from a capillary bed within the oral cavity of the subject as illustrated, for example, in FIGS. 5(a) and (b). The preferred locations are along the top of the nipple 412 (FIGS. 17(a)-(c)), at the tip of the nipple 412 (FIG. 17(d)), and along the bottom of the nipple 412 (FIG. 17(e)). Also, the pulse oximeter elements 20, 22 may be located in and/or along the nipple shank 4126 as illustrated, for example, in FIGS. 17(f)-(i).

The placement and location of the light source 20 and the light detector 22 depicted in FIGS. 5(a)-(b) and 16-18(f) may be switched with respect to each other. Furthermore, the light source 20 and the light detector 22 may be in a variety of exemplary spatial locations relative to each other as shown, for example, in FIGS. 18(a)-(f). Although FIGS. 18(a)-(f) illustrated the pulse oximeter sensor elements 20, 22 on the top of the nipple 412, these elements may have similar spatial locations on other portions of the nipple 412 such as the tip, bottom, and along the shank 4126.

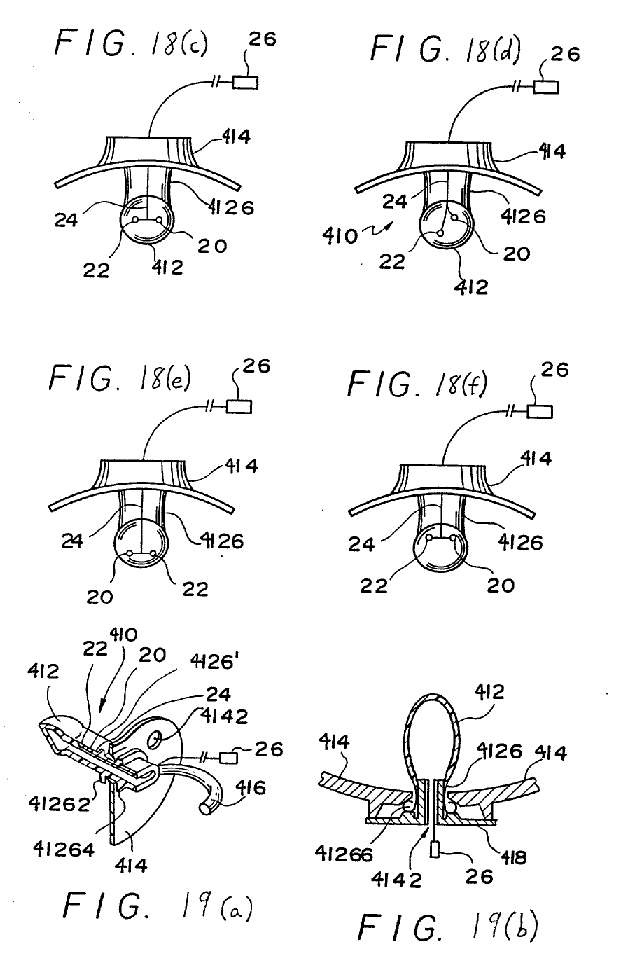

The nipple 412 preferably is attached or mounted to the shield 414. An example of one type of mounting is integrally forming the nipple 412 with the shield 414, for example by mechanically coupling the nipple 412 to the shield 414. Another mounting arrangement, as illustrated in FIG. 19(a), is to have the nipple 412 include a shank 4126′ with two integral spaced collars 41262, 41264 to form a channel to receive the shield 414. Preferably, the shield 414 is at or near the proximal end of the shank 4126′. Preferably to prevent the shield 414 from being pulled off the shank 4126′, a handle 416 is looped through the shank 4126′ as illustrated in FIG. 19(a).

Another example of attaching the nipple 412 to the shield 414 is illustrated in FIG. 19(b). The shield 414 includes an opening for the nipple shank 4126 to pass through preferably such that a rim or section of rolled up material 41266 is located on the proximal side of the shield 414. A plug 418 is inserted into the shield opening 4142 to hold the nipple shank 4126 in place with respect to the shield 414. More preferably, the plug 418 will include a securing mechanism that is compressed as it travels through the shield opening 4142 and then expands on the distal side of the shield 414 to secure the plug 418 in place and hold the nipple 412 securely to the shield 414.

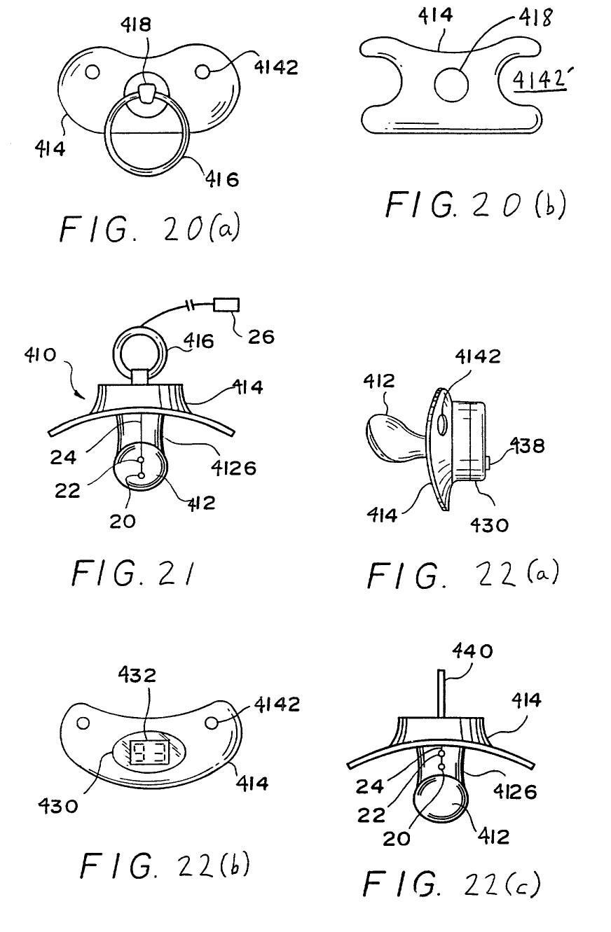

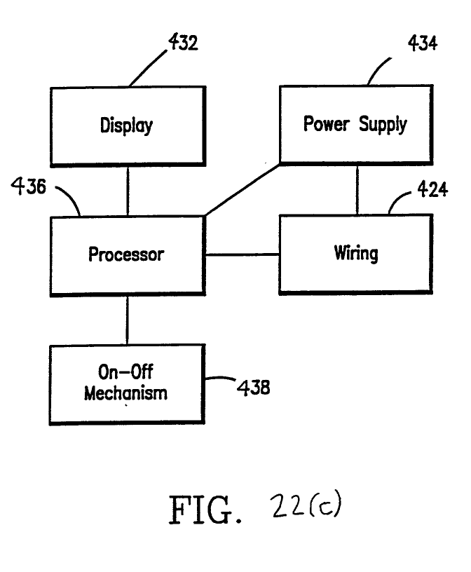

The shield 414 preferably is curved or bowed to form fit to the average baby's face. The shield 414 may be any shape that prevents it from being pulled into the subject's mouth from the suction force placed upon the nipple 412 by the subject. More preferably, the shield 414 will be shaped or include a reference indicator such that the top of the pacifier 410 can be readily determined by looking at the shield 414. In an alternative embodiment, the shield 414 preferably includes a plurality of holes (or relief openings) 4142 to allow for spit to be discharged without interference from the pacifier 410 as illustrated, for example, in FIGS. 20(a) and 22(a)-(b). FIG. 20(b) illustrates a relief opening 4142′ that allows insertion of a catheter such as an endotracheal tube. A further alternative is for the shield to include a mesh pattern over at least a portion of it. Another alternative embodiment adds a ring (or annular or other shaped handle) 416 on the opposite side of the shield 414 from the nipple 412 as illustrated in FIGS. 20(a) and 21 that may attach to either the shield 414 or the nipple 412. Preferably, the ring is hinged, collapsible, and/or flexible.

An alternative embodiment of the pacifier oximeter is the placement of the oximeter signal processing device within a housing 430 extending from the shield 414 on the side opposite the nipple 412 as illustrated, for example, in FIGS. 22(a) and (b). The oximeter signal processing device preferably is a miniature spectrophotometer. The oximeter signal processing device preferably will include a display 432, a power supply (such as a battery) 434, and a processor 436 to perform calculations and to drive the display 432, and an on-off button (or switch/mechanism) 438 as illustrated in FIG. 22(c). The display 432 preferably will show the blood oxygenation level of the subject as illustrated in FIG. 22(b). More preferably, the display 432 is a digital display. The processor 436 preferably will connect to the wiring 24 running from the pulse oximeter sensor elements 20, 22, calculate the blood oxygenation level, and drive both the display 432 and the light source 20. The processor 436 preferably is a circuit that includes an analog circuit and/or an integrated circuit, which is hardwired and/or programmed. Preferably, the display 432, the power supply 434, the processor 436 will reside on a printed circuit board that includes appropriate circuitry and provides a connection to wiring 24.

Another alternative embodiment of the pacifier oximeter is that the light source 20 and the light detector 22 may be in wireless communication with the external device instead of connected with the external cord 26 as illustrated in FIG. 23 as a rod (or antenna or transmitter) 440. Alternatively, the antenna 440 may take the shape as a handle 416 similar to the one illustrated, for example, in FIGS. 20(a) and 21 without the external cord 26. Preferably, the wireless communication will occur through an antenna 440 extending away from the pacifier 410. The transmitter may be incorporated within the antenna 440 or some other housing incorporated into the shield 414. Preferably, the antenna 440 will be sufficiently sturdy to withstand tugging and being played with during use by the subject. This alternative embodiment also preferably includes a power source such as a battery to power all of the electrical components. The power source preferably is located within the shield, a housing, or as part of the antenna 440.

A further alternative embodiment of the pacifier oximeter is to provide a bite block on the distal side of the shield 414 between the shield 414 and the nipple 412. The bite block may be an extension of the shield material or a hardened nipple shank 4126. The flexible nipple 412 preferably is attached to the bite block. Preferably, the bite block will provide a passageway through which the wiring 24 may pass through. The shield 414 and nipple 412 preferably would be shaped such that at least one catheter would have space to enter the oral cavity, for example, for suction and supplying oxygen. This alternative embodiment preferably would be for use during surgery of a variety of subjects other than infants and young children.

The device may be a retrofit of current pacifiers by inserting the pulse oximeter sensor elements from a disposable pulse oximeter like the Nellcor® Oxisensor® II oximeters (Mallinckrodt, Inc., St. Louis, Mo., U.S.A.) by stripping away the packaging and adhesive strip. The ring attached to must pacifiers would be removed leaving access to the interior cavity of the nipple into which the pulse oximeter sensor elements would be inserted such that they faced in the same general direction. The ring then would be reattached.

e. Testing

The method of taking pulse oximeter readings from different surfaces within a patient has been submitted to actual testing in the below-described population and according to the following protocols.

Reflectance Oximetry from the Buccal Surface

The first protocol involved taking readings from the buccal surface. Nine patients were monitored via buccal reflectance pulse oximetry over 20 consecutive surgical procedures, which procedures consisted of burn excision and grafting. Patients ranged in age from 23 to 56 years (Mean=264.8, Standard Deviation (SD)=11.2) and ranged from 17 to 75 percent total body surface area (%TBSA) burned (Mean=274.3%, SD=28.9). Each patient received from one to eight operations (Mean=4.01). Five of these nine patients arrived at the operating room intubated for all of the operations in this study. Four patients were induced and intubated in a standard fashion for all surgical procedures.

A Nellcor® Oxisensor® II D-25 was placed intraoraly between the lower teeth and the left or right buccal surface of the cheek and lip, with the bispectral emitter and detector facing the buccal surface. This pulse oximeter sensor orientation was used for the duration of each case. In addition, a similar disposable oximetric probe was placed on a peripheral digit in the commonly accepted transillumination configuration. At five minute intervals throughout the case, values for both oximetric probes were coded on the anesthesia record.

The differences between the peripheral and buccal SpO₂ (oxygen saturation of hemoglobin) values were insignificant by t-tests for correlated means. Concordance rates as percent agreements were calculated for all cases. Average percent agreement was 84% ranging from 25% to 100%. Three of the 20 samples had percent agreements less than 91%. In each of these cases, the peripheral pulse oximeter sensor appears to have failed, in two cases secondary to sepsis, and in another secondary to peripheral vasoconstriction in the face of a norepinepherine infusion. Buccal SpO₂ readings in all three cases continued to be 97% or greater.

This data suggests that buccal reflectance oximetry is a simple, accurate means of monitoring arterial oxygen saturation in the severely burned patient where oximetric monitoring presents a challenge. Given that central oximetry has been shown in numerous studies to be more rapidly responsive to oxygen saturation variability than peripheral oximetry, as well as more directly reflective of central oxygen saturation, there are few drawbacks and considerable benefit from this method. Indeed, in the three examples in this study where percent agreements were low, the peripheral oximetric probes were returning apparently erratic and/or generally low values while buccal oximetric readings remained at 97% or higher. All three of these patients had peripheral vascular compromise secondary to sepsis and/or a vasoconstricting agent (norepinepherine infusion).

It may appear from the study results, at first blush, that a full range of SpO₂ values was not tested and that the continuously high SpO₂ readings are spurious to the technique. On the contrary, in order to obtain a SpO₂ value greater or less than 85% a very specific set of relationships must be present relative to the bispectral emitter and light sensing oximetric elements. Thus, spuriously high values in particular do not consistently occur. High SpO₂ values require the presence of saturated hemoglobin. Unlike lingual oximetry, this technique is not necessarily limited to intubated patients as a flat disposable oximetric probe could be placed between the cheek and teeth of an awake patient. In addition to operating room considerations, ventilated patients in intensive care settings could benefit from this technique, especially given the more rapid response of a centrally placed pulse oximeter over a peripheral one.

Posterior Pharyngeal Reflectance Oximetry

The second protocol involved comparing posterior pharyngeal reflectance pulse oximetry to conventional peripheral transillumination pulse oximetry in difficult to monitor burn patients. Eight patients' records were reviewed over fourteen consecutive surgical procedures, all consisting of excision and grafting. Patients ranged in age from 9 to 43 years and ranged from 14.5% to 77.5% TBSA burned (Mean=30.4, SD=22.1). The number of operations per patient ranged from one to four.

A Nellcor® Oxisensor® II pulse oximeter probe was placed in the distal lumen of an appropriately sized oropharyngeal airway with sensor and emitter facing the posterior pharynx. A similar probe was placed on a peripheral digit as a transilluminating pulse oximeter. SpO₂ values were noted at five-minute intervals. Concordance statistics as well as a t-test for correlated means were calculated between the simultaneously obtained SpO₂ values.

The mean differences between pharyngeal reflectance and peripheral digital transillumination SpO₂ values were insignificant for all cases. Concordance statistics were as follows: 0.75 (n=1) and 1.0 (n=12).

Given the near perfect concordance statistics in this study, this data suggests that posterior pharyngeal reflectance oximetry is a simple, highly accurate means of monitoring arterial oxygen saturation in the severely burned patient where oximetric monitoring presents a challenge.

Lingual Surface Reflectance Oximetry

The third protocol involved taking readings from the lingual surface. Data was reviewed for eight difficult to monitor patients who were monitored via lingual reflectance pulse oximetry over twenty-five consecutive surgical procedures, all consisting of burn excision and grafting. Patients ranged in age from 26 to 57 years (Mean=36.0, SD=10.3). Patients ranged from 20% to 92% TBSA burned (Mean=66.75%, SD=26.42). Number of operations per patient ranged from one to five (Mean=3.13, SD=1.55). Six of these eight patients arrived at the operating room intubated for all of the operations in this study. Two patients were induced and intubated in a standard fashion.

In each case, a Nellcor® Oxisensor® II D-25 was centered flat on the superior lingual surface with the detector and the bispectral emitter facing the lingual surface. This pulse oximeter configuration was used for the duration of each case. When clinically indicated, an arterial blood gas (ABG) sample was drawn and the SpO₂ noted for clinical monitoring and prior to transfusion in every case. All had multiple ABG's drawn and all patients were transfused. The ABG SaO₂ (oxygen saturation of arterial blood) was noted in each case.

Descriptive statistics and a concordance rate as well as a t-test for correlated means were calculated between the simultaneously obtained SpO₂ and SaO₂ values. The difference between the SpO₂ and SaO₂ values was insignificant by t-test for correlated means (t=1.25, df=24, NS). Upon inspection, the means were very close and the standard deviations were very small, as were the SEM's, all suggesting very little difference or variability between these two measures of oxygen saturation. A concordance rate of 92% was calculated (+1.5%) showing a high degree of relationship between lingual and ABG SaO₂.

This data suggests that lingual reflectance oximetry is a simple, accurate means of monitoring arterial oxygen saturation in the severely burned patient where oximetric monitoring presents a challenge. An existing disposable pulse oximeter sensor was utilized in this study saving the cost of specially designed equipment. Given that central oximetry has been shown to be more rapidly responsive to oxygen saturation variability than peripheral oximetry, there are few drawbacks and considerable benefit from this method. As a practical matter, the foregoing technique is probably limited to intubated patients, as awake, extubated patients could find the presence of a lingual pulse oximeter sensor irritating. However, this limitation would hold with lingual transillumination pulse oximetry as well. In addition to operating room considerations, ventilated patients in intensive care settings could benefit from this technique, especially given the more rapid response of a centrally placed pulse oximeter over a peripheral one.

The preferred and alternative embodiments described above may be combined in a variety of ways with each other.

Although the present invention has been described in terms of particular preferred embodiments, it is not limited to those embodiments. Alternative embodiments, examples, and modifications which would still be encompassed by the invention may be made by those skilled in the art, particularly in light of the foregoing teachings.

Those skilled in the art will appreciate that various adaptations and modifications of the preferred embodiments described above can be configured without departing from the scope and spirit of the invention. Therefore, it is to be understood that, within the scope of the appended claims, the invention may be practiced other than as specifically described herein.

We claim:

1. A reflectance pulse oximeter sensor for use within an intrahead cavity site of a subject comprising:
means for performing reflectance pulse oximetry measurements from a palate, and
means for communicating with an external device.

2. The sensor according to claim 1, wherein the palate is the hard palate.

3. The sensor according to claim 1, wherein the palate is the soft palate.

4. A reflectance pulse oximeter sensor for use within an intrahead cavity site of a subject comprising:
oximeter sensor elements, wherein at least one of said oximeter sensor elements receives reflected light originating with another of said oximeter sensor elements, and
means for causing contact to occur between said oximeter sensor elements and a palate.

5. The sensor according to claim 4, wherein the palate is the hard palate.

6. The sensor according to claim 4, wherein the palate is the soft palate.

7. A reflectance pulse oximeter sensor for use within an intrahead cavity site of a subject comprising:
means for performing reflectance pulse oximetry measurements from a proximal pharyngeal surface, and
means for communicating with an external device.

8. A reflectance pulse oximeter sensor for use within an intrahead cavity site of a subject comprising:
oximeter sensor elements, wherein at least one of said oximeter sensor elements receives reflected light originating with another of said oximeter sensor elements, and
means for causing contact to occur between said oximeter sensor elements and a proximal pharyngeal surface.

9. The sensor according to claim 8, wherein said causing contact means includes a passageway passing therethrough.

10. The sensor according to claim 8, further comprising means for communicating with an external device.

11. A reflectance pulse oximeter sensor for use within an intrahead cavity site of a subject comprising:
means for performing reflectance pulse oximetry measurements from a gingival surface, and
means for communicating with an external device.

12. A reflectance pulse oximeter sensor for use within an intrahead cavity site of a subject comprising:
oximeter sensor elements, wherein at least one of said oximeter sensor elements receives reflected light originating with another of said oximeter sensor elements, and
means for causing contact to occur between said oximeter sensor elements and a gingival surface.

13. A reflectance pulse oximeter sensor for use within a nasal cavity of a subject comprising:
means for performing reflectance pulse oximetry measurements from a capillary bed in the nasal cavity, and
means for communicating with an external device.

14. The method of claim 13, wherein the capillary bed is on a nasal mucosa.

15. The method of claim 13, wherein the capillary bed is on a soft palate

16. The method of claim 13, wherein the capillary bed is on a posterior pharynx.

17. A reflectance pulse oximeter sensor for use within a nasal cavity of a subject comprising:
oximeter sensor elements, wherein at least one of said oximeter sensor elements receives reflected light originating with another of said oximeter sensor elements, and
means for causing contact to occur between said oximeter sensor elements and a capillary bed in the nasal cavity.

18. The method of claim 17, wherein the capillary bed is on a nasal mucosa.

19. The method of claim 17, wherein the capillary bed is on a soft palate

20. The method of claim 17, wherein the capillary bed is on a posterior pharynx.
